(19) United States
(12) Patent Application Publication
(10) Pub. No.: US 2022/0042377 A1
(43) Pub. Date: Feb. 10, 2022

(54) TRACER ASSISTED WEAR DETECTION OF PCD CUTTING ELEMENTS

(71) Applicant: ELEMENT SIX (UK) LIMITED, DIDCOT, OXFORDSHIRE (GB)

(72) Inventors: VALENTINE KANYANTA, DIDCOT (GB); SANTONU GHOSH, DIDCOT (GB)

(73) Assignee: ELEMENT SIX (UK) LIMITED, DIDCOT, OXFORDSHIRE (GB)

(21) Appl. No.: 17/309,715

(22) PCT Filed: Dec. 20, 2019
(86) PCT No.: PCT/EP2019/086685

§ 371 Date: Jun. 16, 2021

(30) Foreign Application Priority Data
Dec. 31, 2018 (GB) 1821328.0

(51) Int. Cl. E21B 12/02; G01N 33/28; E21B 44/00; E21B 10/567

(52) U.S. Cl. CPC E21B 12/02; E21B 10/567; E21B 44/00; G01N 33/2823

(57) ABSTRACT

A cutting element for an earth-boring tool, the cutting element has a substrate; and a body of superhard polycrystalline material bonded to the substrate along an interface. Any one or both of the substrate or the body of superhard polycrystalline material has one or more sealed channels or regions therein, one or more of the regions or channels being arranged to retain a tracer element to provide data relating to a condition of the cutting element.

### FIELD

The present disclosure generally relates to cutting elements for use on or in connection with earth-boring tools such as drill bits, to earth-boring tools including such cutting elements, and to methods of making and using such cutting elements and tools.

### BACKGROUND

In the oil and gas industry, cutting tools such as downhole drill bits, including roller cone bits and fixed cutter bits, are designed and manufactured to minimize the probability of catastrophic drill bit failure during drilling operations. During drilling operations the loss from a drill bit of a roller cone, or a polycrystalline diamond compact acting as a cutter element therein can impede the drilling and may necessitate an expensive and time consuming operation to retrieve the bit or components thereof from the wellbore before catastrophic damage to the drill bit itself occurs.

Conventionally, logging while drilling (LWD) and measuring while drilling (MWD) measurements are obtained from measurements behind the drill head and are therefore off-set from the drill bit itself and the cutting elements therein. While a number of sensors and measurement systems may record information near the earth-boring drill bit, conventional polycrystalline diamond (PCD) cutting elements used in earth-boring drill bits do not provide measurements directly at the drill bit. This off-set of the sensors may contribute to errors in measurements that relate directly to the condition of the cutting elements.

In drilling operations, a cutting element, also termed an insert, is subjected to heavy loads and high temperatures at various stages of its useful life. In the early stages of drilling, when the sharp cutting edge of the insert contacts the subterranean formation, it is subjected to large contact pressures. This results in the possibility of a number of fracture processes such as fatigue cracking being initiated. As the cutting edge of the insert wears, the contact pressure decreases and is generally too low to cause high energy failures. However, this pressure can still propagate cracks initiated under high contact pressures and may eventually result in spalling-type failures. In the drilling industry, PCD cutter performance is determined by a cutter's ability to achieve high penetration rates in increasingly demanding environments, and still retain a good condition post-drilling (enabling re-use if desired). In any drilling application, cutters may wear through a combination of smooth, abrasive type wear and spalling/chipping type wear. Whilst a smooth, abrasive wear mode is desirable because it delivers maximum benefit from the highly wear-resistant PCD material, spalling or chipping type wear is unfavourable. Even fairly minimal fracture damage of this type can have a deleterious effect on both cutting life and performance.

Cutting efficiency may be rapidly reduced by spalling-type wear as the rate of penetration of the drill bit into the formation is slowed. Once chipping begins, the amount of damage to the diamond table continually increases, as a result of the increased normal force required to achieve a given depth of cut. Therefore, as cutter damage occurs and the rate of penetration of the drill bit decreases, the response of increasing weight on bit may quickly lead to further degradation and ultimately catastrophic failure of the chipped cutting element.

PCD cutting elements are typically provided with a theoretical usable lifetime which may be predicted in terms of, for example, time, number of metres cut, number of drilling operations and the like. However, as chipping is a brittle process, the performance of any individual cutting element may greatly exceed that of another individual cutting element, and this effect is difficult to predict which may have an impact on the actual useable lifetime of any individual cutting element.

There is therefore a need to be able to detect parameters during use of the cutting element such as chipping, and wear scar size, and to measure or predict cutting element life more accurately during operation, leading to less risk of damaging the drill bits or tools into which the cutting elements are inserted.

### SUMMARY

According to a first version there is provided a cutting element for an earth-boring tool, the cutting element comprising:
a substrate; and
a body of superhard polycrystalline material bonded to the substrate along an interface; wherein
any one or both of the substrate or the body of superhard polycrystalline material comprises one or more sealed channels or regions therein, one or more of the regions or channels being arranged to retain a tracer element to provide data relating to a condition of the cutting element.

According to a second version there is provided an earth-boring tool, comprising:
a body; and
at least one cutting element defined above attached to the body.

According to a third version there is provided a method of obtaining a measurement at an earth-boring tool, the method comprising:
receiving, during at least one of a borehole drilling operation and a borehole enlarging operation through a subterranean formation, a signal through analysis of drilling fluid that a tracer element has been released from any one or more of recesses or channels in a cutter element attached to the earth boring tool; and
correlating at least one characteristic of the signal with at least one parameter associated with at least one characteristic of the condition of the cutter element.

### BRIEF DESCRIPTION OF THE DRAWINGS

Non-limiting example arrangements to illustrate the present disclosure are described with reference to the accompanying drawings, in which:

FIG. 1a is a schematic cross-sectional side view of a first example cutting element;

FIG. 1b is a schematic top plan view of the cutting element of FIG. 1a;

FIG. 1c is a schematic cross-sectional side view of a second example cutting element;

FIG. 2a is a schematic cross-sectional side view of a further example cutting element; and

FIG. 2b is a top plan view of the cutting element of FIG. 2a.

### DETAILED DESCRIPTION

Referring in general to the following description and accompanying drawings, various versions of the present disclosure are illustrated to show its structure and method of operation. Common elements of the illustrated examples are designated by the same reference numerals.

As used herein, “drill bit” means and includes any type of bit or tool used for drilling during the formation or enlargement of a wellbore in subterranean formations and includes, for example, fixed cutter bits, rotary drill bits, percussion bits, core bits, eccentric bits, bi-center bits, reamers, mills, drag bits, roller cone bits, hybrid bits and other drilling bits and tools known in the art.

As used herein, a “superhard material” is a material having a Vickers hardness of at least about 28 GPa. Diamond and cubic boron nitride (cBN) material are examples of superhard materials.

As used herein, a “superhard construction” means a construction comprising a body of polycrystalline superhard material. In such a construction, a substrate may be attached thereto or the body of polycrystalline material may be free-standing and unbacked.

As used herein, polycrystalline diamond (PCD) is a type of polycrystalline superhard (PCS) material comprising a mass of diamond grains, a substantial portion of which are directly inter-bonded with each other and in which the content of diamond is at least about 80 volume percent of the material. In one example of PCD material, interstices between the diamond grains may be at least partly filled with a binder material comprising a catalyst for diamond. As used herein, “interstices” or “interstitial regions” are regions between the diamond grains of PCD material. In examples of PCD material, some or all interstices or interstitial regions may be substantially or partially filled with a material other than diamond, or they may be substantially empty. PCD material may comprise at least a region from which catalyst material has been removed from the interstices, leaving interstitial voids between the diamond grains.

Cutter elements for use in drill bits in the oil and gas industry typically comprise a layer of polycrystalline diamond (PCD) bonded to a cemented carbide substrate. PCD material is typically made by subjecting an aggregated mass of diamond particles or grains to an ultra-high pressure of greater than about 5 GPa, and temperature of at least about 1200° C., typically about 1440° C., in the presence of a sintering aid, also referred to as a solvent-catalyst material for diamond. Solvent-catalyst materials for diamond are understood to be materials that are capable of promoting direct inter-growth of diamond grains at a pressure and temperature condition at which diamond is thermodynamically more stable than graphite.

Examples of solvent-catalyst materials for diamond are cobalt, iron, nickel and certain alloys including alloys of any of these elements.

As used herein, PCBN (polycrystalline cubic boron nitride) material refers to a type of superhard material comprising grains of cubic boron nitride (cBN) dispersed within a matrix comprising metal or ceramic.

The term “substrate” as used herein means any substrate over which the superhard material layer is formed. For example, a “substrate” as used herein may be a transition layer formed over another substrate.

The superhard construction shown in the figures may be suitable, for example, for use as a cutter insert for a drill bit for boring into the earth. Such an earth-boring drill bit (not shown) includes a plurality of cutting elements, and typically includes a bit body which may be secured to a shank by way of a threaded connection and/or a weld extending around the earth-boring drill bit on an exterior surface thereof along an interface between the bit body and the shank. A plurality of cutting elements are attached to a face of the bit body, one or more of which may comprise a cutting element as described herein in further detail below.

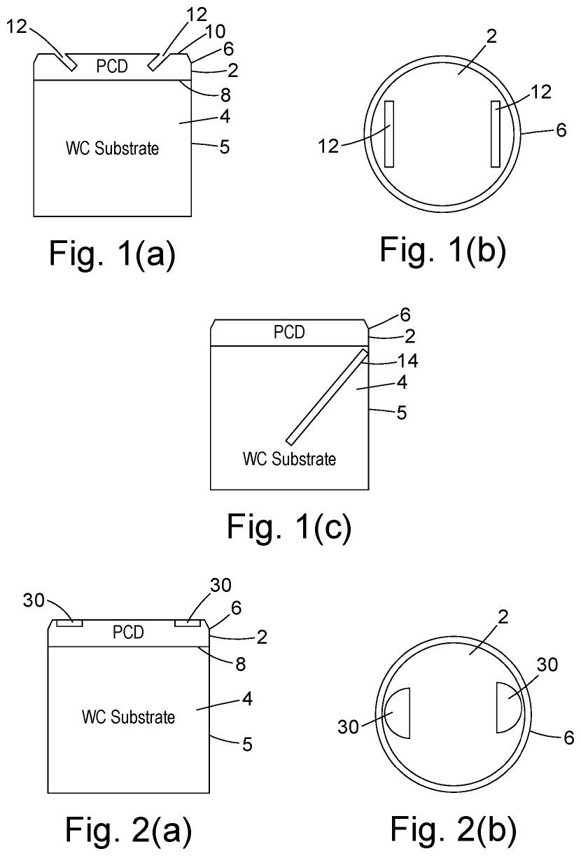

FIGS. 1a and 1b show a first example cutting element for use in a drill bit of the type described above. The cutting element includes a body of superhard material 2, such as polycrystalline diamond material (PCD) comprising for example at least 95 vol % diamond, formed on a substrate 4. The substrate 4 may be formed of a hard material such as cemented tungsten carbide. The cutting element may be mounted into a bit body such as a drag bit body (not shown) for boring into the earth.

The exposed top surface 10 of the body of superhard material 2 opposite the substrate 4 forms the cutting face, also known as the working surface, which is the surface which, along with its edge 6, performs the cutting in use.

At one end of the substrate 4 is an interface surface 8 that forms an interface with the body of superhard material 2 which is attached thereto along this interface surface. As shown in the example of FIG. 1a, the cutting element may, for example, be generally cylindrical.

In the examples where the body of superhard material 2 comprises PCD, the PCD material may be, for example, formed of diamond grains that are of natural and/or synthetic origin.

In the first example shown in FIGS. 1a and 1 b, where FIG. 1b is a plan view of the top of the cutting element of FIG. 1a, one or more regions or channels 12 are formed in the body of superhard material 2 that extend from the working surface 10 into the body of superhard material 2 from a position adjacent the cutting edge 6. The regions or channels 12 may extend into the body of superhard material 2 at any desired orientation and to any desired depth in the body of superhard material 2. The regions or channels 12 form one or more pockets in the body of superhard material 2 into which a tracer element may be introduced and retained. The tracer element(s) may, for example, be a radioactive element such as ⁶⁰Co, ¹⁹²Ir, or ²²⁶Ra gamma emitting isotopes, or a chemical tracer element such as fluorescent diamond or other fluorescent material for which a suitable detector may be chosen to detect release of the element from the channel or recess when it is breached during use. Alternatively, the tracer elements could be in the form of one or more MicroRFID tags which are miniature Radio Frequency Identification (RFID) tags that have very little information embedded in the tag. The MicroRFID tags may in some examples be formed of a copper antenna on a Pyrex™ glass substrate.

The example cutting element shown in FIG. 1c differs from that shown in FIGS. 1a and 1b in that the region or channel 14 containing the tracer element is formed in and extends through the substrate 4 instead of being formed in and extending through the body of superhard material 2. In the example of FIG. 1c, the region or channel 14 extends from a location adjacent the interface 8 with the body of superhard material 2 and adjacent the peripheral side edge 5 of the substrate instead of from the working surface 10 of the body of superhard material 2.

The regions or channels 12, 14 containing the tracer element(s) may be of any desired shape and size and the example of FIGS. 2a and 2b differs from those of FIGS. 1a to 1c in that the two regions 30 in this example are substantially semi-circular in cross-section whereas those of FIGS. 1a to 1 d are substantially rectangular or circular in cross-section. Furthermore, in the example of FIGS. 2a and 2b, the two regions 30 are formed in the working surface 10 of the body of superhard material at a location adjacent the cutting edge 6 on opposing sides of the working surface and extend through the body of superhard material 2 but are spaced from the interface 8 with the substrate 4.

In some examples, any one or more of the one or more channels or regions 12, 14 may have a substantially circular cross-section with a diameter of less than around 2 mm.

In some examples, any one or more of the one or more channels or regions 12, 14 are spaced from a peripheral side surface of the cutting element by a distance of around 0.8 mm or less.

In the examples of FIG. 1c where the one or more channels or regions 14 extend into the substrate, the channels or regions 14 may extend from a position between around 2 to around 8 mm below the interface 8 with the body of superhard material 2.

As known in the art, a body of PCD material 2 may be formed by subjecting diamond particles to high temperature, high pressure (HTHP) conditions in the presence of a metal solvent catalyst (e.g., one or more of cobalt, iron, and nickel).

An example method of preparing the cutting element of FIGS. 1a and 1b is as follows. A pre-sinter mixture was prepared by combining a mass of diamond particles with a non-diamond phase mixture designed to act as a solvent/catalyst for diamond, such as cobalt, and to form up to around 20 wt % in the sintered product. The pre-sinter mixture was loaded into a cup and placed in an HP/HT reaction cell assembly together with a mass of carbide to form the substrate. The contents of the cell assembly were subjected to HP/HT processing. The HP/HT processing conditions selected were sufficient to effect inter-crystalline bonding between adjacent grains of diamond particles and the joining of sintered particles to the cemented metal carbide support to form a PCD construction comprising a PCD structure integrally formed on and bonded to the cemented tungsten carbide substrate. In one example, the processing conditions generally involved the imposition for about 3 to 120 minutes of a temperature of at least about 1200 degrees C. and a super high pressure of greater than about 5 GPa. In some examples, the pre-sinter assembly may be subjected to a pressure of at least about 6 GPa, at least about 6.5 GPa, at least about 7 GPa or even at least about 7.5 GPa or more, at a temperature of around 1440 deg C.

In some examples, both the bodies of, for example, diamond and carbide material plus sintering aid/binder/catalyst are applied as powders and sintered simultaneously in a single UHP/HT process.

In another example, the substrate may be pre-sintered in a separate process before being bonded together in the HP/HT press during sintering of the super hard polycrystalline material.

In some examples, the cemented carbide substrate 4 may be formed of tungsten carbide particles bonded together by the binder material, the binder material comprising an alloy of any one or more of Co, Ni and Cr. The tungsten carbide particles may form at least 70 weight percent and at most 95 weight percent of the substrate.

After sintering, the PCD construction was subjected to further treatment to remove the canister material and to shape the construction to the desired cutting element shape and size. In the example of FIGS. 1a and 1b, the regions or channels 12 into which the tracer elements are to be introduced may be formed by conventional techniques such as electric discharge machining (EDM), grinding, spark eroding, or using a laser or other similar methods to create one or more regions or channels 12 in the working surface 10 of the body of PCD material 2 in a region spaced from but adjacent the cutting edge 6. In the example of FIG. 1c, the region(s), or pocket(s) 14 into which the tracer element is/are to be introduced may be formed in the substrate 4 from a position on the peripheral side edge 5 adjacent the interface 8 with the body of PCD material 2 and extend into the body of the substrate towards the longitudinal axis thereof. These regions 14 in the substrate may be formed, for example, after the sintering process of the cutting element, or in a pre-formed substrate before sintering with the diamond grains to form the cutting element, or in situ through inclusion of a plug that is removed after sintering.

The orientation, depth and dimensions of the recesses, regions or channels 12, 14, 30 may be chosen to suit the desired application and tracer element to be retained therein. In the examples of FIGS. 1a, 1b and 1c, the tracer element(s) may be, for example, radioactive tracer elements and may be prepared as follows. In some examples, the radioactive trace(s) were mixed with an oxide ceramic and further irradiated to increase the activation level. Based on the fluid flow rate and a measurement frequency of 10 minutes, around 0.4 g of this mixture was inserted into the channels 12 in the body of PCD material 2 (in the example of FIGS. 1a and 1b) and in the substrate 4 (in the example of FIG. 1c) and the channels were then sealed with high temperature ceramic glue.

In the examples of FIGS. 1a to 1c, the tracer elements may be radioactive elements such as ⁶⁰Co, ¹⁹²Ir, or ²²⁶Ra gamma emitting isotopes. Alternatively, the tracer elements could be in the form of one or more MicroRFID tags which are miniature Radio Frequency Identification (RFID) tags that have very little information embedded in the tag. For the examples, several MicroRFID MEMS having dimensions of 350 μm×350 μm were packed inside the recesses 12, 14 as shown in FIGS. 1a to 1c. The MicroRFID tags comprised a copper antenna on a Pyrex™ glass substrate.

In the example of FIGS. 2a and 2b, the regions 30 retaining the tracer elements may be formed in the cutting surface 10 of the body of superhard material 2 in a shallow region of the cutting surface 10 adjacent the cutting edge 6. Radioactive material to be used as the tracer element in this example may be captured inside the body of superhard material during its formation in the above described HPHT process by using, for example, radioactive diamond grains as the diamond grains in a region 30 of the PCD material. Any high temperature withstanding isotopes could be captured inside the diamond grains, for example, diamond particles may be manufactured using ¹⁴C isotope. A ¹⁴C rich methane gas source may be used to form ¹⁴C CVD diamond which is then crushed into the desired particle size required to manufacture the PCD material for the regions 30 in the cutter element. To create the regions 30 in the example of FIGS. 2a and 2b, the radioactive diamond may be made into tape using a conventional tape casting technique. This tape may then be cut to the desired shape and size and the pre-sinter assembly loaded into the cup as described above with respect to FIGS. 1a to 1 c, with the tape to form the regions 30 positioned such that the radioactive diamond stays close to the cutting edge in the sintered cutter element. The cutter element is then sintered as described above and processed to the desired shape and size.

The cutting elements of the type shown in FIGS. 1a to 2b may be provided along blades on the face of a drill bit body (not shown). The cutting elements may be secured to the bit body within pockets therein using, for example a conventional brazing process.

During drilling operations, the earth-boring drill bit is positioned at the bottom of a wellbore such that the cutting elements are adjacent the earth formation to be drilled. Equipment such as a rotary table or a top drive may be used to rotate the drill string and the earth-boring drill bit within the wellbore hole. Alternatively, the shank of the earth-boring drill bit may be coupled to the drive shaft of a down-hole motor, which may be used to rotate the earth-boring drill bit. As the earth-boring drill bit is rotated, drilling fluid is pumped to the face of the bit body through a longitudinal bore and internal fluid passageways. The drilling fluid has a generally circulating motion in that it flows from a tank on the surface to the bottom of the hole being drilled by the drill bit and then back to the surface. Rotation of the earth-boring drill bit causes the cutting elements to scrape across and shear away the surface of the underlying formation. The formation cuttings mix with, and are suspended within, the drilling fluid and pass through junk slots and the annular space between the wellbore hole and the drill string to the surface of the earth formation.

In use, the cutting elements shear away the surface of the underlying formation and wear scar forms progressively in the superhard material 2 in the region of the cutting edge 6. As used herein, a “wear scar” is a surface of the cutter formed in use by the removal of a volume of cutter material due to wear of the cutter. As a cutter wears in use, material may progressively be removed from proximate the cutting edge, thereby continually redefining the position and shape of the cutting edge, rake face and flank as the wear scar forms.

Once the wear scar has reached a critical size, for example if it nears the interface 8 between the body of superhard material 2 and the substrate 4, the cutter will fail and will require replacement. To inhibit damage to the expensive drill bit and more efficiently provide the operator with an indication that drilling should be halted to replace one or more cutters in the drill bit ahead of imminent failure of the cutters, it is advantageous to have a means of detecting in real time when the cutter is nearing the end of its working life and the wear scar is reaching its critical size, before catastrophic failure of the cutter, to indicate to the drill bit operator that the drill bit needs to be removed from the well bore and one or more cutters are required to be replaced or spun to present a new cutting edge and working surface to the formation being drilled. The cutter elements of the examples are designed to provide the operator of the drill bit with an indication of the condition of the cutter elements once they have reached a certain wear scar size. Once the wear on the example cutter element reaches the regions or channels 12, 14, 30 in which the tracer elements are contained and the wear scar breaches region(s) or channels(s) 12, 14, 30, or the cutter spalls to the extent that the regions or channels 12, 14, 30 are breached to expose the tracer element, the tracer element will be released into the drilling fluid and will be carried to the surface as the fluid circulates. In some examples such as where a radioactive isotope is used as the tracer element, detection that the tracer element is present in the drilling fluid and therefore that the wear scar has reached a certain size requiring replacement of the cutter element may be performed by a gamma ray detector in the downhole assembly.

In the examples where the tracer element retained in the region 12, 14, 30 is a microRFID tag, once the wear scar or a spall of the cutter element breaks the seal of the region 12, 14, 30 releasing the microRFID tag into the drilling fluid, it may be possible to detect this event by, for example, an RFID detector located either on the surface or on the drill bit which indicates to the operator that the microRFID tag is no longer located in the cutter element. For example, if a sample of the drilling fluid is periodically collected, for example at 10 minute intervals, and passed through a microfluidic channel where a receiver antenna with a frequency range of, for example, between around 840-900 MHz, is placed at the bottom of the channel, the antenna may detect and identify the MicroRFID tag and thereby the cutter which requires replacing.

Similarly, where the tracer element is, for example, a radioactive tracer, once released from its recess in the cutter element, the tracer will travel to the surface with the drilling fluid and if fluid samples are periodically collected and tested, for example at 10 minute intervals and a quantity of the tracer element such as ¹⁴C is detected, this will provide an indication to the operator of the wear on the cutter(s) suggesting that the wear scar has reached a size indicating the cutting element should be replaced.

In some examples, individual cutters may have different tracer elements such as individual MicroRFID tags associated with each cutter to enable unique identification of the specific cutter during the drilling operation.

In some examples, the cutting elements may have a generally cylindrical shape. In other examples, the cutting elements be a different shape, such as conical, or ovoid.

In some examples, the body of PCD material may be formed as a standalone object, that is, a free-standing unbacked body of PCD material, and may be attached to a substrate in a subsequent step.

In the example of where the tracer element is a MicroRFID tag, the tag may transmit a signal away from the cutting element to a receiver which may be connected to a processor that may be part of a data collection module located in the earth-boring drill bit, the bit shank, or in other instrumentation in the bottom hole assembly, or to equipment located above the surface of the formation.

It will therefore be seen that various versions of the present disclosure include cutting elements and methods of forming same for earth-boring drill bits which may provide an indication of the wear of the cutting elements obtained directly from locations at the drill bit to which they are mounted and used. The cutting elements may be used to identify real-time information on the wear of the cutting elements which may assist in reducing the risk of loss or damage to the cutting elements and/or the earth-boring drill bit to which the cutting elements are mounted.

Although the foregoing description contains many specifics, these are not to be construed as limiting the scope of the present disclosure, but merely as providing certain exemplary versions.

1. A cutting element for an earth-boring tool, the cutting element comprising:
a substrate; and
a body of superhard polycrystalline material bonded to the substrate along an interface; wherein
any one or both of the substrate or the body of superhard polycrystalline material comprises one or more sealed channels or regions therein, one or more of the regions or channels being arranged to retain a tracer element to provide data relating to a condition of the cutting element.

2. The cutting element of claim 1, wherein the body of superhard polycrystalline material comprises any one or more of polycrystalline diamond, diamond-like carbon, or cubic boron nitride of natural and/or synthetic origin.

3. The cutting element of claim 1, wherein the tracer element comprises any one or more of a radioactive isotope, a microRFID tag, a chemical tracer element, a fluorescent material, or radioactive diamond particles or grains.

4. The cutting element of claim 1, wherein any one or more of the one or more regions or channels extend(s) from a cutting surface into the body of superhard polycrystalline material.

5. The cutting element of claim 4, wherein the body of superhard material has a cutting edge formed by the intersection of the cutting surface and peripheral side surface of the cutting element or by a chamfer extending therebetween, the cutting edge being the intersection of the chamfer with the peripheral side surface, any one or more of the one or more regions or channels extending from the cutting surface adjacent the cutting edge and into the body of superhard polycrystalline material.

6. The cutting element of claim 1, wherein any one or more of the one or more regions or channels extend(s) from a peripheral side surface of the substrate into substrate.

7. The cutting element of claim 1, wherein any one or more of the one or more regions or channels extend(s) through the substrate from a region adjacent the interface and adjacent a peripheral side surface of the substrate.

8. The cutting element of claim 7, wherein any one or more of the one or more channels or regions extends into the substrate from a position between around 2 mm to around 8 mm below the interface with the body of superhard material.

9. The cutting element of claim 1, wherein any one or more of the one or more regions or channels extend(s) at an inclined angle to the plane along which the longitudinal axis of the cutter element extends.

10. The cutting element of claim 1, wherein any one or more of the one or more regions or channels extend(s) in a plane substantially parallel to the plane along which the longitudinal axis of the cutter element extends.

11. (canceled)

12. The cutting element of claim 1, wherein the body of superhard polycrystalline material comprises polycrystalline diamond material having inter-bonded diamond grains with interstitial spaces between the inter-bonded diamond grains, at least a portion of the interstitial spaces being substantially free of metal solvent catalyst material.

13. The cutting element of claim 1, wherein any one or more of the one or more channels or regions have a substantially circular cross-section with a diameter of less than around 2 mm.

14. The cutting element of claim 1, wherein any one or more of the one or more channels or regions are spaced from a peripheral side surface of the cutting element by a distance of around 0.8 mm or less.

15. An earth-boring tool, comprising:
a body; and
at least one cutting element according to claim 1 attached to the body.

16. The earth-boring tool of claim 15, further comprising a detector arranged to detect the release of the tracer element from any one or more of the regions or channels.

17. A method of obtaining a measurement at an earth-boring tool, the method comprising:
receiving, during at least one of a borehole drilling operation and a borehole enlarging operation through a subterranean formation, a signal through analysis of drilling fluid that a tracer element has been released from any one or more of recesses or channels in a cutter element attached to the earth boring tool; and
correlating at least one characteristic of the signal with at least one parameter associated with at least one characteristic of the condition of the cutter element.

18. The method of claim 17, wherein correlating the at least one characteristic of the condition of the cutting element with the at least one characteristic of the signal comprises correlating the size of a wear scar on the cutting element.

19. The method of claim 17, further comprising actively controlling the at least one of a borehole drilling operation and a borehole enlarging operation through the subterranean formation responsive to data derived from the signal.

20. The method of claim 17, wherein the step of receiving the signal comprises detecting using a gamma ray sensor a radioactive a radioactive material, the radioactive material forming the tracer element from the cutting element.

21. The method of claim 17, wherein the step of receiving the signal comprises detecting the presence or absence of a signal from an RFID tag, the RFID tag forming the tracer element from the cutting element.
